(19) United States
(12) Patent Application Publication
(10) Pub. No.: US 2025/0050009 A1
(43) Pub. Date: Feb. 13, 2025

(54) REUSABLE BOTTLE CAP FOR NEGATIVE PRESSURE WOUND THERAPY

(71) Applicants: Jonathan HAAR, Cambridge, MA (US); Bruce HOFFMAN, Smithfield, UT (US)

(72) Inventors: Jonathan HAAR, Cambridge, MA (US); Bruce HOFFMAN, Smithfield, UT (US)

(21) Appl. No.: 18/796,994

(22) Filed: Aug. 7, 2024

Related U.S. Application Data
(60) Provisional application No. 63/517,974, filed on Aug. 7, 2023.

(51) Int. Cl. A61M 1/00

(52) U.S. Cl. CPC A61M 1/98

(57) ABSTRACT

A cap configured to be removably coupled with a receptacle for performing negative pressure wound therapy for a patient. The cap comprises a base section configured to be fluidly coupled with both a negative pressure source and a wound of the patient. The cap further comprises a coupling section extending from the base section, with the coupling section being configured to engage with a mouth of the receptacle. The cap includes an injection conduit extending beyond the coupling section, such that when the cap is mounted on the receptacle, the injection conduit is configured to be positioned within an interior of the receptacle.

### CROSS-REFERENCE TO RELATED APPLICATIONS

The present patent application claims the priority benefit of U.S. Provisional Patent Application Ser. No. 63/517,974, filed Aug. 7, 2023, entitled “REUSABLE BOTTLE CAP FOR NEGATIVE PRESSURE WOUND THERAPY AND GENERAL HOSPITAL SUCTION USE,” which is incorporated by reference in its entirety herein.

### FIELD OF THE INVENTION

Embodiments of the present invention are directed to systems and methods for negative pressure wound therapy. More particularly, embodiments of the present invention are directed to systems and methods for a reusable cap that can be used in cooperation with common receptacles, such as bottles, to collect fluid from a patient's wound during negative pressure wound therapy.

### BACKGROUND OF THE INVENTION

Typically, in a modern hospital setting, vacuum systems are incorporated directly into the building structure of the hospital. Vacuum valves are located in each patient room and in the operating rooms. As such, to obtain vacuum or suction functionality, all that is needed is vacuum tubing that can be connected to the vacuum valves. For negative pressure wound therapy, a vacuum container can be connected to the vacuum tubing to collect fluid from a patient's wound.

Unfortunately, in resource-poor areas of the world, medical facilities generally do not have any such built-in vacuum systems. As such, expensive suction canisters must be used with portable vacuum pumps. These suction canisters must be imported at great expense because of the shipping volume the canisters take up.

As such, it would be beneficial if there were a system for negative pressure wound therapy that could be used with commonly-used receptacles, such as plastic or glass drink bottles. Furthermore, it would be beneficial if such a system could be sterilized and reused, to reduce cost and expense, particularly in resource-poor areas.

### SUMMARY OF THE INVENTION

In one embodiment of the present invention, there is provided a cap configured to be removably coupled with a receptacle for performing negative pressure wound therapy for a patient. The cap comprises a base section configured to be fluidly coupled with both a negative pressure source and a wound of the patient. The cap further comprises a coupling section extending from the base section, with the coupling section being configured to engage with a mouth of the receptacle. The cap includes an injection conduit extending beyond the coupling section, such that when the cap is mounted on the receptacle, the injection conduit is configured to be positioned within an interior of the receptacle. It is noted that the injection conduit extending down beyond the coupling section is beneficial so as to not create an unwanted feedback loop whereby fluid from a patient's wound could be sucked back up into the negative pressure source rendering the source non-sterile.

In another embodiment of the present invention there is provided a system for performing negative pressure wound therapy for a patient. The system comprises a negative pressure source and a cap. The cap comprises a base section configured to be fluidly coupled with both the negative pressure source and a wound of the patient. The cap further comprises a coupling section extending from the base section. The coupling section is configured to engage with a mouth of a receptacle. The cap includes an injection conduit extending beyond the coupling section, such that when the cap is mounted on the receptacle, the injection conduit is configured to be positioned within an interior of the receptacle.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE FIGURES

Embodiments of the present invention are described herein with reference to the following drawing figures, wherein:

FIG. 1 is a schematic diagram of a system according to embodiments of the present invention for performing negative pressure wound therapy for a patient;

FIG. 2A is a perspective view of an embodiment of a cap from the system of FIG. 1, which can be reused with receptacles for performing negative pressure wound therapy for a patient;

FIG. 2B is a side elevation view of the cap from FIG. 2A;

FIG. 2C is a top plan view of the cap from FIGS. 2A and 2B;

FIG. 2D is a vertical cross-section of the cap from FIGS. 2A-2C;

FIG. 2E is a bottom perspective view of the cap from FIGS. 2A-2D, particularly illustrating the cap not having threads;

FIG. 3A is a perspective view of another embodiment of a cap from the system of FIG. 1, which can be reused with receptacles for performing negative pressure wound therapy for a patient;

FIG. 3B is a side elevation view of the cap from FIG. 3A;

FIG. 3C is a top plan view of the cap from FIGS. 3A and 3B;

FIG. 3D is a vertical cross-section of the cap from FIGS. 3A-3C; and

FIG. 3E is a bottom perspective view of the cap from FIGS. 3A-3D, particularly illustrating the cap having threads.

The figures are not intended to limit the present invention to the specific embodiments they depict. While the drawings do not necessarily provide exact dimensions or tolerances for the illustrated structures or components, the drawings are to scale with respect to the relationships between the components of the structures illustrated in the drawings.

### DETAILED DESCRIPTION

The following detailed description of the present invention references various embodiments. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment,” “an embodiment,” or “embodiments” mean that the feature or features referred to are included in at least one embodiment of the invention. Separate references to “one embodiment,” “an embodiment,” or “embodiments” in this description do not necessarily refer to the same embodiment and are not mutually exclusive unless so stated. Specifically, a feature, component, action, step, etc. described in one embodiment may also be included in other embodiments, but is not necessarily included. Thus, particular implementations of the present invention can include a variety of combinations and/or integrations of the embodiments described herein.

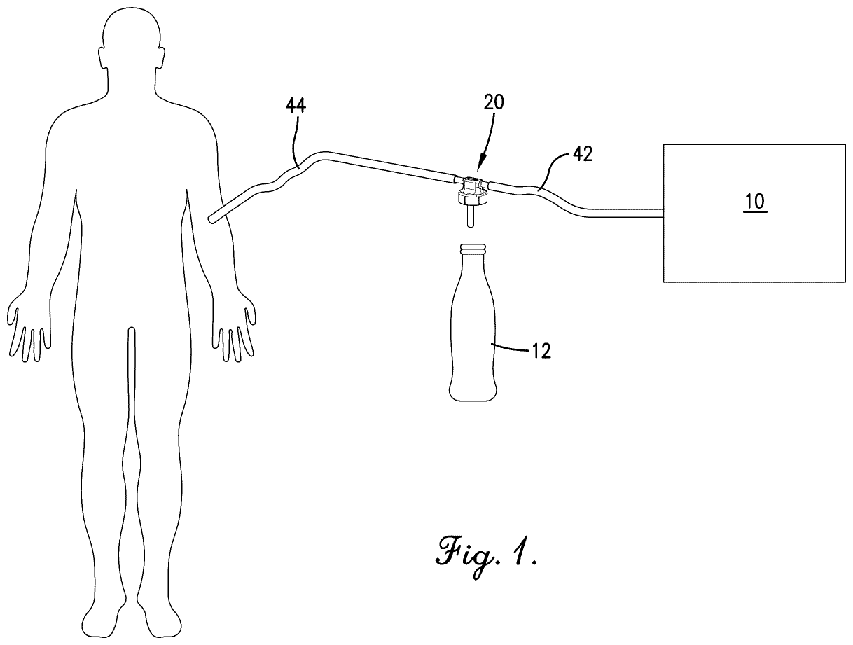

Broadly embodiments of the present invention are directed to systems and methods for negative pressure wound therapy. An exemplary system is shown in FIG. 1 and broadly comprises a negative pressure (“NP”) source 10, a receptable 12, and a cap 20. The NP source 10 may comprise generally any device configured to generate suction or vacuum pressure. For example, the NP source 10 may be portable and may comprise an electrical pump configured to generate a sub-atmospheric pressure. As such, the NP source 10 may be powered by batteries or by a building's mains power. The NP source 10 may be configured to operate in either continuous and intermittent modes of operation and may be controllable to adjust the specific degree of sub-atmospheric pressure generated (e.g., within a range of 40-80 mm Hg sub-atmospheric pressure). The receptacle 12 may comprise generally any type of receptacle configured to hold liquid. However, in many preferred configurations of the system, the receptacle 12 will comprise common drink receptacles, such as glass or plastic soda or beer bottles having mouths with openings for adding or removing liquid into/out of the bottles.

The cap 20 will be described in more detail below. Broadly, however, as illustrated in FIG. 1, the cap 20 is configured to be removably coupled with the mouth of the receptacle 12 to cover the primary opening of the receptacle 12. The cap 20 will also be configured to be fluidly connected to the NP source 10 via a first section of tubing connected to and extending between the cap 20 and the NP source 10. The cap 20 will further be configured to be fluidly connected to a wound of a patient via a second section of tubing connected to and extending between the cap 20 and the patient. As such, with the NP source 10 generating a suction pressure, such suction pressure will extend to the cap 20, via the first section of tubing, and into the receptacle 12. The suction will further extend through the second section of tubing to the wound of the patient. such that liquid from the patient's wound can be drawn through the second section of tubing to the cap 20 and deposited into the receptacle 12. Once the receptacle 12 has been filled with liquid from the wound of the patient, the receptacle 12 can be detached from the cap 20 and can be disposed. If necessary, another receptacle 12 can be attached to the cap 20, such that additional liquid from the patient's wound can be collected. Alternatively, the original receptacle 12 can be emptied, cleaned, and re-used.

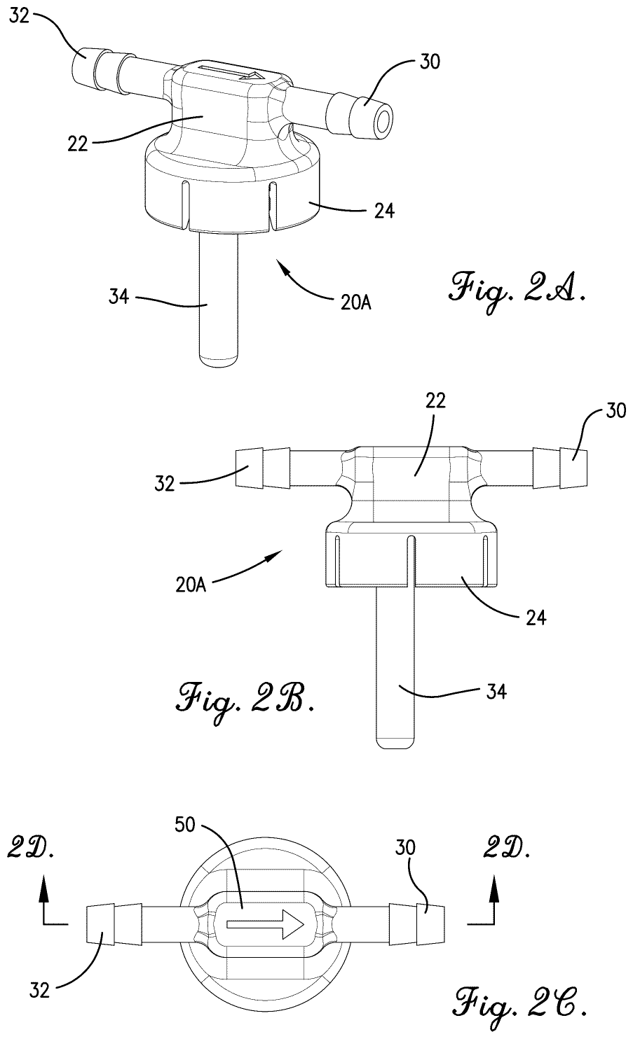

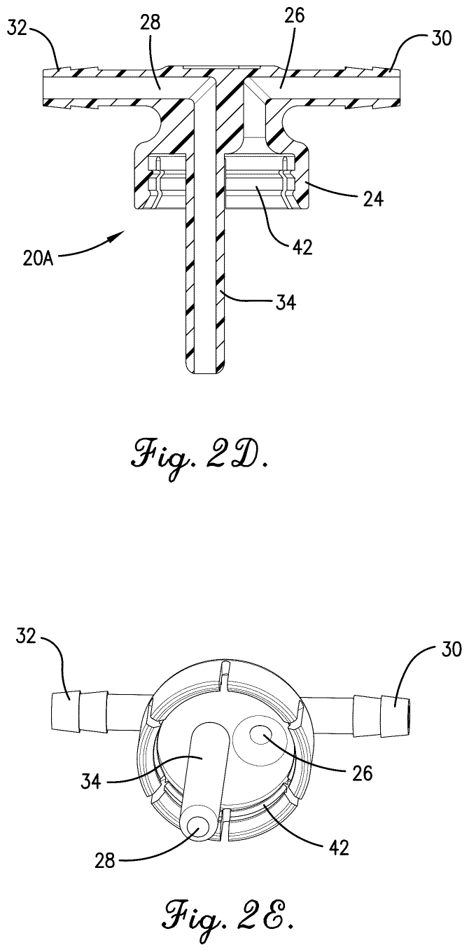

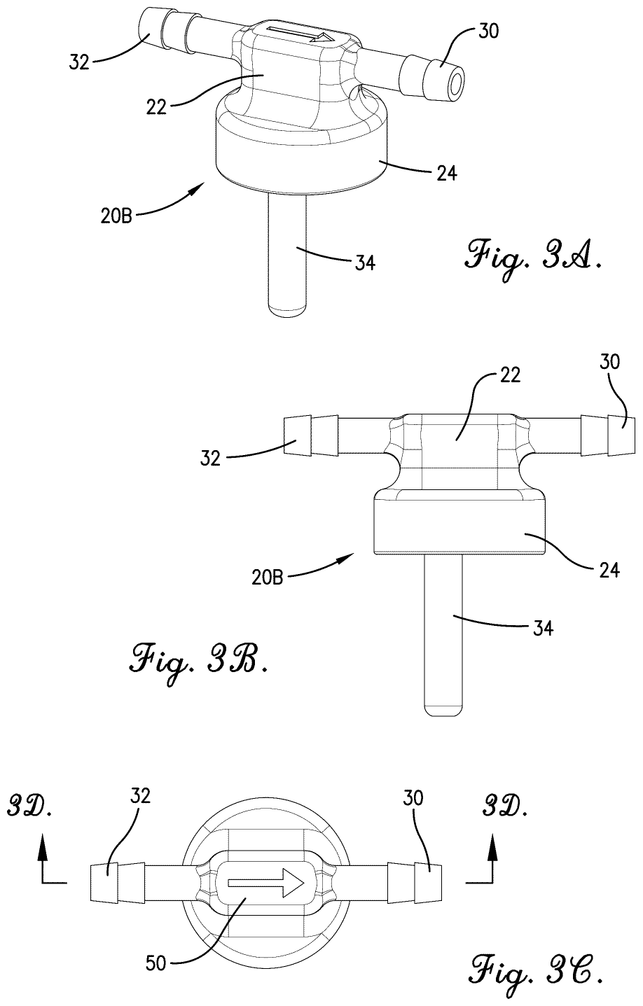

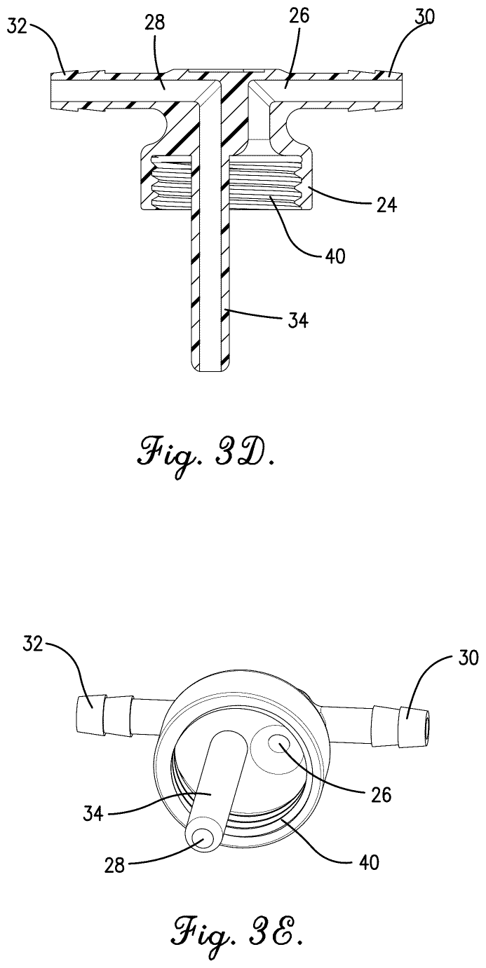

Relational and/or directional terms, such as “above”, “below”, “up”, “upper”, “upward”, “down”, “downward”, “lower”, “top”, “bottom”, “outer”, “inner”, “front”, “forward,” “back”, “rear”, “rearward”, etc., along with orientation terms, such as “horizontal” and “vertical”, may be used throughout this description. These terms retain their commonly accepted definitions and are used with reference to embodiments of the technology and the positions, directions, and orientations thereof shown in the accompanying figures. For example, with reference to FIGS. 2B and 3B, the top of the cap 20 is shown in the upper portion of the figures, while the bottom of the cap 20 is shown in the lower portion of the figures.

Turning to the cap 20 in more detail, FIGS. 2A-2E illustrate a first embodiment of a cap 20A, whereas FIGS. 3A-3E illustrate a second embodiment of a cap 20B. The caps 20A and 20B are configured generally the same, except that cap 20B includes threads to permit the cap 20B to be secured to a receptacle 12 in a threaded manner, whereas the cap 20A does not include threads but is configured for a snap-fit connection with a receptacle 12.

More generally, the cap 20 according to embodiment of the present invention may comprise a base section 22 and a coupling section 24 extending from the base section 22. The base section 22 may comprise a pair of channels, as illustrated in FIGS. 2D and 3D, formed therethrough, including a vacuum channel 26 and a liquid channel 28. The base section 22 may additionally comprise a plurality of conduits extending from a main portion of the base section 22. For example, a vacuum conduit 30 may extend from the main portion of the of the base section 22 and may form part of, or otherwise be fluidly coupled with, the vacuum channel 26. In addition, a liquid conduit 32 may extend from the main portion of the of the base section 22 and may form part of, or otherwise be fluidly coupled with, the liquid channel 28. In some embodiments, the vacuum conduit 30 and the liquid conduit 32 may extend in opposite directions from each other (e.g., 180° apart).

In more detail, a first end of the vacuum channel 26 (e.g., an upper end) may connect with the vacuum conduit 30, such that the vacuum conduit 30 extends generally horizontally outward from the first end of the vacuum channel 26. From the vacuum conduit 30, the vacuum channel 26 extends generally horizontally inward into the main portion of the base section 22 and then changes direction approximately 90°, such that the vacuum channel 26 extends downward to a second end of the vacuum channel 26 (e.g., a lower end). The second end of the vacuum channel 26 may present an opening, as shown in FIGS. 2E and 3E, formed through a bottom of the base section 22. As a result, when the cap 20 is positioned on the mouth of a receptacle 12 (covering the opening in the receptacle 12), the vacuum channel 26 and the vacuum conduit 30 are fluidly connected with the interior of the receptacle 12.

As perhaps best shown in FIGS. 2D and 3D, the vacuum channel 26 and the vacuum conduit 30 are interconnected in a way to form a continuous channel or pathway. As such, the vacuum channel 26 and the vacuum conduit 30 may collectively form the vacuum channel 26.

Similarly, a first end of the liquid channel 28 (e.g., an upper end) may connect with the liquid conduit 32, such that the liquid conduit 32 extends generally horizontally outward from the liquid channel 28. From the liquid conduit 32, the liquid channel 28 extends generally horizontally inward into the main portion of the base section 22 and then changes direction approximately 90°, such that the liquid channel 28 extends downward to a second end of the liquid channel 28 (e.g., a lower end). The second end of the liquid channel 28 may be positioned at the bottom of the base section 22 and may connect with an injection conduit 34. The injection conduit 34 (being fluidly connected with the liquid channel 28) may extend downward from the bottom of the base section 22 generally vertically. As a result, when the cap 20 is positioned on the opening of a receptacle 12, the injection conduit 34 is positioned within the interior of the receptacle 12, such that the injection conduit 34, the liquid channel 28, and the liquid conduit 32 are all fluidly connected with the interior of the receptacle 12.

As perhaps best shown in FIGS. 2D and 3D, the injection conduit 34, the liquid channel 28, and the liquid conduit 32 are interconnected in a way to form a continuous channel or pathway. As such, the injection conduit 34, the liquid channel 28, and the liquid conduit 32 may collectively form the liquid channel 28.

The coupling section 24 of the cap 20 comprises a cylindrical sidewall that extends down from the bottom of the base section 22. In some embodiments, such as with the cap 20B shown in FIG. 3E, the interior surface of the coupling section 24 may include threads 40. As such, the cap 20B may be threadedly secured to a receptacle 12 with threads positioned around the exterior of a mouth of the receptacle. In contrast, the cap 20A, as shown in FIG. 2E, may not include threads on the interior surface of the coupling section 24. Instead, the interior surface of the coupling section 24 of the cap 20A may have an annular groove 42 formed therein near the bottom of the base section 22 where the coupling section 24 is joined to the base section 22. In such embodiments (with the cap 20 not having threads), the sidewall of the coupling section 24 may be formed into a plurality of individual sections divided by vertical gaps (see, e.g., FIG. 2B). The individual sections of the sidewall may, thus, flex inward and outward with respect to the base section 22. As such, the cap 20A may be snap fit onto a receptacle 12, such as a glass bottle, that does not have threads positioned around the exterior of the mouth of the receptacle 12. Specifically, such glass bottles commonly have an annular rib around the tip of the opening the bottle. Such annular rib can fit within the annular groove 42 formed in the coupling section 24 to facilitate a snap-fit, sealed connection between the cap 20A and the receptacle 12. In some embodiments, the cap 20B with threads may also include an annular groove (similar to annular groove 42 shown in cap 20A), which assists in providing a sealed connection between the cap 20B and the receptacle 12. In some embodiments, a sealing aid, such as Teflon tape, may be used between the cap 20 and the receptacle 12 to enhance the fluid seal between the cap 20 and the receptacle 12.

The cap 20 may be formed from various types of materials. However, in some embodiments, it may be preferable for the cap 20 to be formed from a sterilizable material, such as Nylon_12. As such, the cap 20 can be cleaned, sterilized, and reused. For example, the cap 20 may be heated in an autoclave prior to use. The cap 20 may be formed by 3D printing, cast molding, injection molding, or the like. The cap 20 may be formed in various sizes, as necessary to be coupled with the mouths of various sizes of receptacles. As noted previously, the cap 20 will preferably be used with plastic or glass drink bottles (e.g., soda or beer bottles). As such, the cap 20 will commonly be formed with a coupling section 24 having a width (or diameter) from 1 to 4 inches, from 1.25 to 3 inches, or from 1.5 to 2.5 inches, or from 1.5 to 2.0 inches.

One particularly notable feature of the cap 20 is that the injection conduit 34 extends down a significant distance from the bottom of the base section 22. Specifically, the free, lower end of the injection conduit 34 may extend downward to a position below the second end of the vacuum channel 26 (presented as an opening in the bottom of the base section 22). As such, when the cap 20 is coupled with a receptacle 12, liquid injected into the interior of the bottle from the injection conduit 34 will not be inadvertently suctioned up into the vacuum channel 26 and back to the NP source 10 where the liquid may clog or otherwise damage the NP source 10. In some specific embodiments, the injection conduit 34 may have a length from 1 to 6 inches, from 2 to 5 inches, from 2 to 4 inches, or about 3 inches. In some embodiments, the length of the injection conduit 34 may form a ratio with a width of the coupling section 24 of at least 1:1, 1.25:1, 1.5:1, 1.75:1, or 2:1.

In view of the above, the system for negative pressure wound therapy may function as follows. First, the cap 20 may be sterilized, as previously described. Next, the cap 20 will be coupled with a receptacle 12. The cap 20 will be secured onto the mouth of the receptacle 12, such that the cap 20 covers the opening of the receptacle. As such, the injection conduit 34 extends down within the interior of the receptacle 12, while the second end of the vacuum channel 26 remains fluidly connected with the interior of the receptacle 12. As discussed above, the cap 20A can be snap-fit on the mouth of a receptacle 12 not having threads. In contrast, the cap 20B can be threaded on the mouth of a receptacle 12 that has threads. Regardless, the cap 20 will be securely coupled with the receptacle 12, and in some embodiments, a rubber o-ring or other gasket may be positioned between the cap 20 and the receptacle 12 to enhance the liquid seal between the cap 20 and the receptacle 12. Next, as illustrated in FIG. 1, a first section of tubing 42 may have a first end engaged with the free end of the vacuum conduit 30 and a second end engaged with the NP source 10. In addition, a second section of tubing 44 may have a first end engaged with the free end of the liquid conduit 32 and a second end provided to a wound of a patient. Such tubing 42, 44 may be formed from medical-grade silicon or other similar material. In some embodiments, as shown in FIGS. 2C and 3C, a top of the cap 20 may be formed (e.g., imprinted) with an arrow 50 that shows the direction of airflow of the system or the direction of the NP source 10. As such, the first section of tubing 42 that is intended to interconnect the cap 20 and the NP source 10 can easily be connected to the conduit that is adjacent to a head of the arrow 50 (i.e., the vacuum conduit 30).

Thus, methods for performing negative pressure wound therapy for a patient are also described herein. To begin operation, the NP source 10 is powered on, which generates a negative pressure, or suction, through the first section of tubing 42, through the vacuum conduit 30, through the vacuum channel 26 of the cap 20, within the receptacle 12, through the injection conduit 34, through the liquid channel 28 of the cap 20, through the liquid conduit 32, and through the second section of tubing 44. As such, with the second end of the second section of tubing 44 engaged with the wound of the patient, biological fluid and/or other liquid from the patient, such as blood, serum, extracellular fluid, serosanguineous drainage, or other wound exudate, can be suctioned from the wound, through the second section of tubing 44, through the liquid conduit 32, through the liquid channel 28 of the cap 20, through the injection conduit 34, and into the receptacle 12 where the liquid can be retained.

Once the receptacle 12 has been filled with liquid from the patient, the receptable 12 can be removed from the cap 20, and another receptacle 12 can be attached to the cap 20 to collect more liquid from the patient's wound. Generally, the receptacle 12 will be considered full when the level of liquid within the receptacle has reached the level of the free, lower end of the injection conduit 34. In some instance, it may be permissible to empty a receptacle 12 of liquid from the patient's wound and then re-attach the same receptacle 12 to the cap 20 to collect more liquid from the patient instead of attaching an entirely new receptacle 12 to the cap 20.

It is noted that the system may be used in methods for performing negative pressure wound therapy for a patient in remote areas, such as in resource-poor areas of the world. For example, the cap 20 may be used with various receptacles to capture fluid from the wounds of patients using suction provided by an NP device 10. Nevertheless, it should be understood that the system may also be used within medical facilities, e.g., for general hospital operations such as surgical suction in a surgical suite as long as enough receptacles 12 are present for the type of operation.

Having thus described one or more embodiments of the invention, what is claimed as new and desired to be protected by Letters Patent includes the following:

1. A cap configured to be removably coupled with a receptacle for performing negative pressure wound therapy for a patient, wherein said cap comprises:
a base section configured to be fluidly coupled with both a negative pressure source and a wound of the patient;
a coupling section extending from the base section, wherein the coupling section is configured to engage with a mouth of the receptacle,
wherein the cap includes an injection conduit extending beyond the coupling section, such that when the cap is mounted on the receptacle the injection conduit is configured to be positioned within an interior of the receptacle.

2. The cap of claim 1, wherein an interior surface of the coupling section includes threads, such that the cap is configured to be threadedly engaged with the receptacle.

3. The cap of claim 2, wherein the receptacle is a plastic bottle.

4. The cap from claim 1, wherein an interior surface of the coupling section does not include threads, and wherein the cap is configured to be snap-fit onto the mouth of the receptacle.

5. The cap of claim 4, wherein the receptacle is a glass bottle.

6. The cap of claim 1, wherein the cap is formed from a sterilizable material.

7. The cap of claim 6, wherein the sterilizable material is Nylon_12.

8. The cap of claim 1, wherein the cap is formed by 3D printing, cast molding, or injection molding.

9. The cap of claim 1, wherein the base section includes a liquid conduit configured to be coupled with a tube that extends between the wound of the patient and the cap.

10. The cap of claim 9, wherein the base section includes a liquid channel formed through the base section, and wherein the liquid conduit, the liquid channel, and the injection conduit are all fluidly connected, such that the cap is configured to permit liquid from the wound of the patient to flow from the tube that extends between the wound of the patient and the cap, through the cap, and into the receptacle.

11. The cap of claim 1, wherein the base section includes a vacuum conduit configured to be coupled with a tube that extends between the negative pressure source and the cap.

12. The cap of claim 11, wherein the base section includes a vacuum channel formed through the base section, and wherein the vacuum conduit and the vacuum channel are fluidly connected, such that the cap is configured to permit a vacuum pressure generated by the negative pressure source to extend through the tube, through the cap, and into the receptacle.

13. The cap of claim 1, wherein the base section includes a vacuum channel formed through the base section through which a vacuum pressure generated by the negative pressure source is configured to extend, and wherein a lower end of the injection conduit extends lower than a lower end the vacuum channel.

14. A system for performing negative pressure wound therapy for a patient, wherein said system comprises:
a negative pressure source; and
a cap, wherein the cap comprises—
a base section configured to be fluidly coupled with both the negative pressure source and a wound of the patient;
a coupling section extending from the base section, wherein the coupling section is configured to engage with a mouth of a receptacle,
wherein the cap includes an injection conduit extending beyond the coupling section, such that when the cap is mounted on the receptacle the injection conduit is configured to be positioned within an interior of the receptacle.

15. The system of claim 14, wherein the negative pressure source comprises a vacuum pump.

16. The system of claim 14, wherein the receptacle is a plastic or glass bottle, and wherein the cap is configured to be threadedly-engaged with or snap-fit onto the receptacle.

17. The system of claim 14, wherein the cap is formed from a sterilizable material.

18. The system of claim 14, wherein the base section includes a liquid conduit configured to be coupled with a tube that extends between the wound of the patient and the cap, wherein the base section includes a liquid channel formed through the base section, and wherein the liquid conduit, the liquid channel, and the injection conduit are all fluidly connected, such that the cap is configured to permit liquid from the wound of the patient to flow from the tube that extends between the wound of the patient and the cap, through the cap, and into the receptacle.

19. The system of claim 14, wherein the base section includes a vacuum conduit configured to be coupled with a tube that extends between the negative pressure source and the cap, wherein the base section includes a vacuum channel formed through the base section, and wherein the vacuum conduit and the vacuum channel are all fluidly connected, such that the cap is configured to permit a vacuum pressure generated by the negative pressure source to extend through the tube, through the cap, and into the receptacle.

20. The system of claim 14, wherein the base section includes a vacuum channel formed through the base section through which a vacuum pressure generated by the negative pressure source is configured to extend, and wherein a lower end of the injection conduit extends lower than a lower end the vacuum channel.
